(19) United States
(12) Patent Application Publication
(10) Pub. No.: US 2025/0264479 A1
(43) Pub. Date: Aug. 21, 2025

(54) FUNCTIONAL ASSAY FOR QUICKLY DETERMINING IMMUNE STATUS

(71) Applicant: bioMérieux, Marcy-l'Étoile (FR)

(72) Inventors: Franck BERTHIER, Saint Georges de Reneins (FR); Soizic DANIEL, Trévoux (FR); Sandrine DUCROT, Porte des Pierres Dorées (FR)

(21) Appl. No.: 18/875,839

(22) PCT Filed: Jun. 26, 2023
(86) PCT No.: PCT/FR2023/000126

§ 371 Date: Dec. 17, 2024

(30) Foreign Application Priority Data
Jun. 29, 2022 (FR) 2206512
Mar. 30, 2023 (FR) 2303122

(51) Int. Cl. G01N 33/68; G01N 33/50

(52) U.S. Cl. CPC G01N 33/6866; G01N 33/5091; G01N 2333/42; G01N 2333/57

(57) ABSTRACT

The present invention relates to a method for quickly determining the immune status of an individual. The method comprises the following steps: taking a volume of a whole blood sample from the individual; stimulating the whole blood sample by incubating the latter with a quantity of phytohemagglutinin (PHA) at a temperature between 35° C. and 39° C. for a minimum of 3 to 3.5 hours; evaluating the level of interferon-gamma production induced by this incubation/stimulation, the evaluated level subsequently providing an indication of the individual's immune status.

The present invention relates to the evaluation and determination of an individual's immune status. More particularly, it relates to methods and tools dedicated to measuring the overall level of an individual's cell-mediated immunity, and whose operating principle is that of a functional immune assay.

By proposing a method and clinical tools that are capable of allowing reliable and rapid evaluation of a patient's immune status and/or diagnosis of any dysfunction or imbalance in his or her immune response (immune deficiency or hyperactivity), the invention is advantageously positioned as a valuable aid proposed to clinicians in their decision-making.

An individual's immune status corresponds to the functional state of his or her immune system, i.e. the ability of the body to defend itself against potentially hazardous agents. These defense and protection mechanisms are mainly deployed against pathogens of infectious origin and exogenous to the body, for instance microorganisms—such as viruses, bacteria, fungi and protozoa. They may also be deployed against endogenous agents, notably cells transformed as a result of physical and/or chemical damage (as may be the case for infected cells, cancer cells or senescent cells).

The immune system's response to attack by a potentially hazardous agent, whether exogenous or endogenous, is a dynamic phenomenon which, when it is correctly suited, enables the organism's integrity to be maintained. Conversely, a weakened, insufficient or unbalanced immune response exposes the body to a high risk of developing pathologies. A weak or ineffective immune response thus favors opportunistic infections, the onset of sepsis and/or viral reactivations, while an exacerbated immune response may account for the onset of allergies, autoimmune diseases (for example, multiple sclerosis, type 1 diabetes, lupus, autoimmune thyroiditis, rheumatoid arthritis, ankylosing spondylitis, Goujerot-Sjögren syndrome, Crohn's disease).

Being able to determine a patient's immune status and/or to monitor its evolution is consequently a major clinical challenge. In this respect, numerous examples of clinical applications may be mentioned, in particular:
identifying patients with a possible immune deficiency (chronic or acute, acquired or induced) and, where appropriate, being able to:
   provide suitable care and medical support; and/or
   prevent intolerance to live attenuated vaccines or drugs contraindicated in cases of immunodeficiency;
monitoring changes in the immune status of patients undergoing immunosuppressive therapy, for example candidates for solid organ transplantation, newly transplanted patients; this would make it possible to best adapt the dosage of immunosuppressive drugs used, in order to establish a level of immunity deemed appropriate to prevent the risk of graft rejection, while minimizing the risk of infection, reactivation of oncogenic viruses and inhibition of patients' antitumor immunity;
monitoring the reconstitution of patients' immune systems after immunosuppressive therapy, to ensure that the process runs smoothly;
monitoring the impact of chemotherapy on a patient's immune status, and thus allowing an optional readjustment or change in therapy; or
managing the treatment of and monitoring patients receiving immunotherapy (notably such as CAR-T (Chimeric Antigenic Receptor-T) cell treatment and treatments based on injection of antibodies, known as anti-checkpoint antibodies).

Being able to determine an individual's immune status and monitor its evolution is also of great interest to the pharmaceutical industry and fundamental research into human health. In this respect, numerous examples of applications may be mentioned, in particular:
in the context of drug development, where its impact on the immune system needs to be evaluated;
in the context of vaccine development, for example, to assess its effects on a possible polarization of the immune response toward a Th1 and/or Th2 type response; or
to assess the possible impact of a pathology or environmental factor on an individual's immune system.

Among the methods currently known to be capable of determining and/or evaluating an individual's immune status, mention may be made firstly of the lymphocyte proliferation test (LPT) and the lymphoblastic transformation test (LTT), which are designed to quantify lymphocyte proliferation following stimulation with mitogens (for example, lectins such as phytohemagglutinin (PHA), concanavalin A (conA) and Pokeweed mitogen (PWM)) or pathogen-specific antigens. These tests are particularly time-consuming to perform. In particular, after isolation, mononuclear cells must be subjected to stimulation for 3 to 7 days. The cells are then recovered and DNA replication or cell division is measured by flow cytometry, by means of tracer incorporation.

Performed much more rapidly, HLA-DR assay by flow cytometry allows measurement of the expression of HLA-DR (for “human leukocyte antigen-D related”) on the surface of monocytes; low expression of this marker is a sign of immune system failure. Similarly, in patients suffering from septic shock, persistent low monocyte HLA-DR expression level is generally a sign of poor survival.

At present, this method of assaying HLA-DR can only be performed using flow cytometry, and few health care centers or medical analysis laboratories have the appropriate equipment. As a result, such a method of determining immune status is more suitable for observational studies and exploratory research than for use in clinical diagnosis. Furthermore, it is cumbersome and difficult to perform, and remains very complicated to normalize/standardize; temperature and cell storage period before labeling, as well as lysis of red blood cells, are all factors with a strong impact on measurement variability, and must therefore be finely controlled (Finck et al., 2003—“Standardisation de la mesure de l'antigène HLA-DR monocytaire par cytométrie en flux: résultat préliminaire et application dans le suivi des chocs septiques [Standardization of monocyte HLA-DR antigen measurement by flow cytometry: preliminary results and application in the monitoring of septic shock]”—Ann. Biol. Clin. 2003, 61:441-448).

Requiring more accessible equipment and less complicated to perform, methods for determining immune status, known as IFA (Immune Functional Assays), are based on the measurement of cellular activity (involving one or more types of immune cells—lymphocytes, macrophages, monocytes, dendritic cells, granulocytes) in response to a particular stimulus. Depending on the nature of the stimulant(s) used for this purpose, the level of immunity studied is either a specific level of immunity, i.e. an immune response specifically deployed and directed against a given target pathogen, or an overall level of immunity which reflects the general state of the individual's immune system. In both cases, measurement of said cellular activity consists in assaying one or more cytokines whose expression is modulated by stimulation (for example IFNγ, TNFα, interleukins, etc.).

To determine a specific level of immunity, the stimulant(s) used generally reproduce epitope (protein and/or glycoside) units from the target pathogen (for example, for an immune status optionally directed against Mycobacterium tuberculosis, all or part of the protein sequence of markers such as ESAT-6, CFP-10 and TB7.7 is frequently used for stimulation). For the determination of an overall level of immunity, one or more “non-specific” stimulants are used. By way of example, mention may be made of protein kinase A (PKA), phorbol myristate acetate (phorbol-12-myristate-13-acetate or PMA), PHA, conA, Staphylococcal Enterotoxin B (SEB) or lipopolysaccharide (LPS), but also cytokines such as interleukins IL-1, IL-2 and IL-12. Anti-CD3 (or more rarely anti-CD2) monoclonal antibodies are also used, such as OKT-3 with or without anti-CD28.

The present invention focuses more specifically on functional immunoassays dedicated to determining an individual's overall level of cellular immunity, as is the case with the ImmuKnow® (Cylex Inc., USA) and QuantiFERON Monitor® (Qiagen GmbH, Germany) assays, both of which are commercially available.

The ImmuKnow® assay, proposed for immunological monitoring of patients on immunosuppressive drugs after organ transplantation, is designed to identify situations of under-and overdosage. The principle of this assay is based on assaying the intracellular ATP (adenosine triphosphate) synthesized by stimulated CD4⁺ T lymphocytes. The level of intracellular ATP thus measured is supposed to correlate with the patient's overall lymphocyte activity. An activity level identified as low thus indicates an overdose of immunosuppressant and a risk of infection for the patient, whereas an activity level identified as high indicates an underdose of immunosuppressant and a risk of graft rejection.

The ImmuKnow® assay is performed by stimulating a whole blood sample for 15 to 18 hours with a mitogen, in this case PHA. CD4⁺ T lymphocytes are then purified and lyzed to extract the ATP. The latter is finally measured quantitatively by bioluminescence using a luciferin/luciferase system (Stewart, 2012—“ImmuKnow as an immune monitoring tool following organ transplantation”-Le Courrier de la Transplantation, 2012, vol. VII No. 1).

As regards the QuantiFERON Monitor® assay, Douglas et al., 2020 (“The QuantiFERON Monitor® assay is predictive of infection post allogeneic hematopoietic cell transplantation”—Transplant Infectious Disease, 2020, 22 (3): 1-9) describes its use in predicting the risk of infection in patients who have undergone allogeneic hematopoietic stem cell transplantation. For this purpose, a heparinized whole blood sample is stimulated at 37° C. for 16 to 24 hours, using an active agent composition, called QFM LyoSphere™. This composition contains an R848 reagent and a TLR7 receptor agonist, to stimulate the patient's innate immunity, and also an anti-CD3 antibody to stimulate adaptive immunity. After 16 to 24 hours of stimulation, the plasma is collected and its gamma interferon (IFNγ) content is measured. The latter provides an indication of the patient's immune status, in this case an indication of his or her overall level of cell-mediated immunity. This assay takes into account both innate immunity and adaptive immunity components.

Like the ImmuKnow® assay, the QuantiFERON Monitor® assay is particularly time-consuming to perform, the excessive duration being mainly due to the stimulation phase, which alone takes over 15-16 hours.

Since immunodeficient patients often require particular clinical and/or therapeutic management to cope with their high susceptibility to infection, screening for immunodeficiency may be of an urgent nature in many clinical situations, for example:
on admission to a care center,
before surgery,
before prescribing drugs/treatments contraindicated for immunocompromised patients,
in cases of sepsis, where particularly close monitoring is required,
there is thus a real need for clinicians to be able to have access to a diagnostic/prognostic assay for an immunodeficiency state, which can be performed rapidly and which is capable of delivering a result in the shortest possible time.

The object of the present invention is thus to propose a functional immune assay that is capable of enabling determination and evaluation of a patient's immune status in a significantly reduced time compared with functional immune assays that are currently commercially available.

In more general terms, the object of the present invention is to propose a process for in vitro or ex vivo determination of an individual's immune status, the implementation of which is intended to be simple, rapid and performable with technical equipment accessible to healthcare centers and medical analysis laboratories.

The present invention meets all the abovementioned objects. Before presenting its features and distinctive characteristics in greater detail, the following definitions are given to allow a better understanding.

In the context of the present description, the term “determining/evaluating immune status” is used to indicate the ability of an individual's body to mount an immune response to defend and protect itself against potentially hazardous agents. In a very similar manner to “immune status”, the term “level of immunity” may also be used, interchangeably.

The immune status determined/evaluated by means of the present invention may be reported by means of a value, which may be numerical or categorical, and which results directly or indirectly from a measurement of IFNγ produced in response to a stimulation produced in accordance with the present invention. A result given in the form of a numerical value then corresponds to a discrete or continuous variable, representing the level of immunity. A result given in the form of a category value may, for example, combine an individual's immune status with qualifiers such as “normal”, “low” or “high”. Such an indication results from an interpretation/extrapolation based on the level of IFNγ production measured after stimulation and/or a comparison of this level with one or more reference IFNγ expression levels.

The term “whole blood sample” means a venous blood sample, obtained from a sample taken from an individual/patient and consisting essentially of erythrocytes, leukocytes, platelets and plasma. Apart from the possible addition of an anticoagulant, optional dilution and/or possible storage between 2° C. and 8° C., the whole blood sample directly subjected to the process of the invention has not undergone any other treatment, in particular any pretreatment liable to significantly modify its composition (in terms of constituents and proportions between constituents).

The term “evaluate the level of IFNγ production”, in this case the production induced by a stimulation performed in accordance with the present invention, does not necessarily mean measuring with more or less precision the amount of IFNγ that is actually and specifically produced/secreted in response to the stimulation. Such an evaluation may in fact consist of a reasoned estimate of indicators/parameters such as:
the total concentration/amount of IFNγ found in the reaction mixture [whole blood-stimulation solution], or in a sub-fraction of this mixture;
the amount of mRNA transcribed in response to the IFNγ stimulation, and which, apart from a few factors and/or approximations, give a valid account of the IFNγ production thus studied.

Finally, the term “individual” denotes a human being, whatever his or her state of health. A “healthy individual” for the purposes of the present invention is an individual who apparently has no immune system disorders. The term “patient” denotes an individual in contact with a healthcare professional—such as a doctor (for example, a general practitioner)—and/or a medical facility (for instance a hospital emergency or intensive care unit), or else a medical analysis laboratory.

The present invention thus relates to a process for determining an individual's immune status; which comprises the following steps:
providing a volume of whole blood sample from said individual;
stimulating said whole blood sample by incubating it with an amount of phytohemagglutinin (PHA), at a temperature of between 35° C. and 39° C., for a minimum period of 3 hours, for example 3 hours to 8 hours;
evaluating the level of IFNγ production induced; said level thus evaluated gives an indication of the immune status of said individual.

According to a particular embodiment, the stimulation time does not exceed 8 hours and, preferably, it does not exceed 6 hours.

The inventors have thus developed a reliable functional immune assay which can give a result in a particularly short time. Specifically, and contrary to all expectation, the inventors have succeeded in significantly reducing the duration of the stimulation step. All of this was made possible essentially by virtue of the following findings and demonstrations:
1) stimulation of whole blood with PHA brings about a cell-mediated immune response, reflected by IFNγ production; 3 to 4 hours of stimulation are enough to induce IFNγ production that is sufficiently intense to be quantified, including by assay methods and equipment readily accessible to healthcare centers and medical analysis laboratories;
2) PHA stimulation, even of short duration (3 to 4 hours), induces IFNγ production that ranges according to the functional state of the immune system of individuals;
3) PHA-induced IFNγ production is sufficiently sensitive to variations in the functional state of the immune system for the difference between two particular functional states to be reflected by a difference in IFNγ production, readily measurable including via the technical means commonly available to healthcare centers and medical analysis laboratories.

IFNγ production in response to PHA stimulation of whole blood is thus seen as a parameter of choice for developing a system for stratifying the overall state of the immune system of individuals. The process for determining immune status according to the invention advantageously allows the immune status of immunodeficient individuals to be distinguished from that of healthy patients. It also allows different levels of immunocompetence to be distinguished in healthy individuals, and different levels of immunodeficiency in immunosuppressed patients.

According to the invention, the biological sample tested is a whole blood sample. Unlike other blood fractions, it contains all the leukocytes, erythrocytes, platelets and plasma. As a result, PHA-stimulable cells and cells expressing IFNγ in response to PHA stimulation benefit from a relatively well-preserved cellular and biochemical environment, in which physiological interactions between the different cell populations involved in the immune response remain possible. The process of determining immune status according to the invention thus advantageously takes into account the full complexity of the intra-and intercellular mechanisms of the cell-mediated immune response, and can also be applied to individuals/patients under the influence of a medicinal or environmental active agent with immunomodulatory effects.

Advantageously and according to the invention, the whole blood sample is venous blood, collected by the venous route. According to the invention, prior to performing the process according to the invention, the sample has not undergone any treatment other than the possible addition of an anticoagulant and/or dilution.

According to the invention, the whole blood sample is subjected to the stimulation step (equivalently, it may also be referred to as an incubation step or a stimulation/incubation step) within 32 hours of collection. After collection and until the process of the invention is performed, the whole blood sample is stored at between 2° C. and 8° C.

Advantageously and according to the invention, the whole blood sample has been treated with an anticoagulant, preferably immediately after collection.

Advantageously and according to the invention, the whole blood sample has been heparinized (for example treated with lithium heparin).

According to the invention, the whole blood sample is subjected to a stimulation/incubation step with phytohemagglutinin (PHA), a lectin synthesized by plants and particularly known for its mitogenic action on T lymphocytes.

Advantageously and according to the invention, the stimulation/incubation step (equivalently, it may also be referred to as an incubation step) is performed with phytohemagglutinin P (PHA-P).

Advantageously and according to the invention, the stimulation/incubation step is performed with PHA, in particular with PHA-P, in an amount at least equal to 20 μg per mL of whole blood. According to a preferred embodiment, the amount of PHA, in particular of PHA-P, is of the order of 40 μg per mL of whole blood.

Also and according to the invention, the stimulation/incubation step is performed at a temperature of between 35° C. and 39° C. Advantageously and according to the invention, this temperature is 37° C.

As regards the duration of the stimulation/incubation step, this is at least equal to 3 hours and does not exceed 8 hours. Preferably, it is between 3 hours 30 minutes and 6 hours. Even more preferably, the minimum stimulation/incubation time is 3 hours 30 minutes.

According to a particularly preferred embodiment, the level of IFNγ production induced by PHA stimulation is assessed by measuring the IFNγ present in the reaction mixture, the latter consisting of the whole blood sample to which PHA has been added-for example in the form of a PHA solution.

According to one embodiment variant, the level of IFNγ production induced by PHA stimulation is evaluated by assaying the IFNγ present in the liquid fraction of the reaction mixture. To this end, once the stimulation/incubation step has been completed, the liquid fraction is recovered from the reaction mixture, optionally after a decantation or centrifugation step.

According to a preferred embodiment, the level of stimulation-induced IFNγ production is evaluated by performing an IFNγ assay via an immunoassay technique.

Immunoassay methods are widely known to those skilled in the art. For example, the assay may be of the enzyme immunoassay (EIA, Enzyme ImmunoAssay) type, i.e. an immunoassay in which the interaction between binding partner and target analyte is revealed by substrate hydrolysis (enzyme-catalyzed hydrolysis), and the release of a readily detectable and measurable lysis product. Detection and measurement of the lysis product thus reveal the presence and concentration of the target analyte in the test sample.

Depending on the nature of enzyme substrate chosen for assaying, enzymatic hydrolysis releases a colorimetric (ELISA, for Enzyme-Linked Immunosorbent Assay), fluorescent (ELFA, for Enzyme Linked Fluorescent Assay) or chemiluminescent (CLIA, for Chemiluminescence ImmunoAssay) lysis product, which is detectable and which has a readily measurable intensity.

Advantageously and according to the invention, the IFNγ production is evaluated by assaying IFNγ using an ELFA test.

In this particular context and for the purposes of the present description, the term “immunoassay” is to be understood in a broad sense. Strictly speaking, it does not refer solely to techniques for detecting and/or quantifying a target analyte, whose operating principle is based on antigen-antibody recognition and coupling, which as a result requires the use of tools of an immune nature or origin, such as antibodies or antibody fragments (fragments of the Fab, Fab′, F(ab′)₂, scFv (“single-chain fragment variable”) and dsFv (“double-stranded fragment variable”) type). It refers more generally to techniques for the detection and/or quantification of a target analyte, in which antibodies or any other functionally analogous compounds, not necessarily of immune nature or origin, may be used as binding partners in a process of recognition and coupling to the target analyte (or ligand).

In this respect, as examples of binding partners for the purposes of performing an IFNγ immunoassay in the sense of the present invention, mention may be made of:
binding partners of immunological nature or origin, such as anti-IFNγ antibodies (monoclonal or polyclonal), or fragments of these antibodies (such as Fab, Fab′, F(ab′)₂ fragments, scFv (“single-chain fragment variable”) and dsFv (“double-stranded fragment variable”));
non-immunological binding partners such as the IFNγ receptor or a fragment of this receptor that is capable of recognizing and binding IFNγ, or even oligonucleotides, nanofitins, aptamers, DARPins (Designed Ankyrin Repeat ProteINS) or any other synthetic molecule that could recognize and bind IFNγ.

Thus, advantageously and according to the invention, stimulation-induced IFNγ production is evaluated by means of an immunoassay method. This may be quantitative or semiquantitative.

Nonlimiting examples of immunoassay instruments suitable for performing the present invention include instruments from the VIDAS® range (bioMérieux, France), the Simoa® HD-1 (Quanterix, USA), Cobas® or Elecsys® (Roche Diagnostic, Switzerland), LIAISON® (DiaSorin, Italy), Architect® (Abbott, USA), Access 2 (Beckman Coulter, USA), Clarity™ (Singulex, USA) and Vitros® (Johnson & Johnson, USA).

According to a particular embodiment of the process of the invention, this also comprises measurement of basal IFNγ levels. This measurement is performed under the same conditions as for a determination of stimulation-induced IFNγ production performed in accordance with the present invention, except that the whole blood sample is not subjected to any stimulation. In other words, prior to IFNγ assay, the whole blood sample is incubated under the same conditions as a stimulated blood sample, notably in terms of temperature and incubation time, but in the absence of PHA and any other stimulant. This particular IFNγ measurement, which may be used as a control measurement, allows the production of a value in connection with the basal IFNγ level, specific to the whole blood sample analyzed.

Advantageously, the process of determining immune status according to the invention comprises an additional result rendering step, according to which said result is delivered in the form of an indication chosen from:
at least one discrete numerical value reflecting the level of immunity of the individual/patient tested, said at least one numerical value corresponding to:
the value of the assay of IFNγ produced in response to PHA stimulation;
the difference between the value of the assay of IFNγ produced in response to PHA stimulation and the basal IFNγ level measured; and/or
a ratio between the value of the assay of IFNγ produced in response to PHA stimulation and the basal IFNγ level measured;
at least one category value deduced from the comparison between at least one of the numerical values previously listed and at least one reference value:
said at least one reference value having been previously determined from a whole blood sample from the same individual/patient but taken at a different time; the process according to the invention thus provides an indication as to the evolution over time of the immune status of said individual/patient, and/or as to the impact of any treatment on his or her immune system; and/or
said at least one reference value having been previously determined from a set of whole blood samples collected from a population of individuals sharing the same particularity in terms of their immune system (for example, a population of healthy individuals, a population of immunosuppressed individuals); the process according to the invention thus provides an indication as to whether said individual/patient belongs to the reference population and/or his or her immune positioning in relation to this reference population.

Beyond the identification of the immune status of an individual/patient, the process according to the invention has many important clinical applications. Thus, according to another aspect, the present invention relates to the use of a process for determining the immune status of an individual/patient according to the invention, for at least one of the following particular and specific applications:
detecting any immune deficiency;
monitoring the evolution of the immune status of a patient undergoing immunosuppressive therapy;
monitoring the reconstitution of a patient's immune system after immunosuppressive therapy;
monitoring the impact of chemotherapy on a patient's immune status, and thus allowing for any readjustments or changes in therapy;
studying an active agent and its possible impact on the immune system;
studying an environmental factor and its possible impact on the immune system;
detecting and/or studying an infectious agent and its possible impact on the immune system;
diagnosing and/or studying a disease and its possible impact on the immune system.

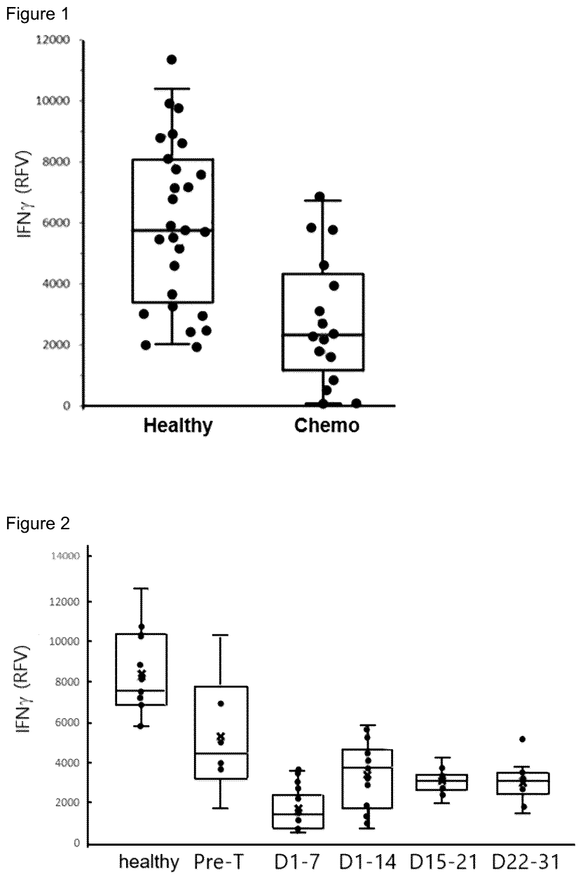

Other aims, features and advantages of the invention will emerge in the light of the detailed description that follows and from the examples developed hereinbelow. These examples refer to the attached FIGS. 1 to 4, which show, in the form of a box diagram, the results of various implementations of a process performed according to the invention.

### EXAMPLES

### Example 1: Stimulation of Whole Blood rom Healthy Donors and Patients Undergoing Chemotherapy

### Origin of the Blood Samples Analyzed

Of the whole blood samples used in this example, a first batch came from 27 healthy, adult individuals who, in principle, showed no symptoms of immunodeficiency. The samples of this first batch were collected by the Etablissements Français du Sang (EFS).

Similarly, a second batch of blood samples was collected from 16 patients undergoing chemotherapy. These samples were collected in hospitals.

Each of the whole blood samples was collected in sterile Vacutainer® tubes (Becton-Dickinson) containing lithium heparin, and then stored upright at 2-8° C., pending performance of the process of the invention.

### Sample Stimulation

For each whole blood sample, after homogenization, 300 μL are collected and transferred to a well of a VIDAS® strip (bioMérieux, France). 300 μL of a PBS-diluted PHA-P solution at 40 μg/mL (Medicago AB, Sweden) are then added.

Similarly, in a second well, in order to determine the basal IFNγ level, 300 μL of PBS buffer (without PHA-P) are added to 300 μL of whole blood.

The reaction mixtures are then incubated for 3 hours 30 minutes at a temperature of 37° C., with controlled evaporation. The stimulation/incubation step is undertaken here using an immunoassay instrument, the VIDAS® 3 system.

### Assaying the INFγ Produced After Stimulation

On conclusion of the 3 hours 30 minutes of stimulation/incubation, 90 μL of the liquid fraction of the reaction mixture are collected and the IFNγ content is measured. For this purpose, the assay part of a VIDAS® TB-IGRA kit, operating on the principle of an ELFA test, is used. Similarly, the VIDAS® IFNγ RUO kit may be used for the same purpose.

### Results

The results obtained are collated in Table 1 hereinbelow, in which INFγ production after stimulation with PHA-P (and without stimulation), is expressed in terms of the fluorescence intensity recorded (RFV, for “Relative Fluorescence Value”) and the estimated IFNγ concentration.

TABLE 1
IFNγ assay without and after PHA-P stimulation, on whole
blood from healthy donors and patients undergoing chemotherapy.

| | | Basal level of IFNγ | | IFNγ after stimulation | |
|---|---|---|---|---|---|
| | Samples | (RFV) | (IU/mL) | (RFV) | (IU/mL) |
| Healthy | s001 | 21 | <0.08 | 7 130 | >8.00 |
| individuals | s002 | 14 | <0.08 | 5 279 | >8.00 |
| | s003 | 14 | <0.08 | 9 823 | >8.00 |
| | s004 | 7 | <0.08 | 3 322 | 6.97 |
| | s005 | 40 | <0.08 | 8 873 | >8.00 |
| | s006 | 14 | <0.08 | 11 396 | >8.00 |
| | s007 | 23 | <0.08 | 2 479 | 4.89 |
| | s008 | 26 | <0.08 | 8 947 | >8.00 |
| | s009 | 17 | <0.08 | 3 054 | 6.27 |
| | s010 | 26 | <0.08 | 9 925 | >8.00 |
| | s011 | 730 | 1.31 | 8 126 | >8.00 |
| | s012 | 15 | <0.08 | 5 635 | >8.00 |
| | s013 | 8 | <0.08 | 6 838 | >8.00 |
| | s014 | 7 | <0.08 | 5 596 | >8.00 |
| | s015 | 13 | <0.08 | 5 752 | >8.00 |
| | s016 | 37 | <0.08 | 2 014 | 3.67 |
| | s017 | 21 | <0.08 | 7 177 | >8.00 |
| | s018 | 12 | <0.08 | 7 595 | >8.00 |
| | s019 | 22 | <0.08 | 4 663 | >8.00 |
| | s020 | 22 | <0.08 | 5 871 | >8.00 |
| | s021 | 57 | <0.08 | 8 667 | >8.00 |
| | s022 | 71 | 0.12 | 5 746 | >8.00 |
| | s023 | 23 | <0.08 | 2 542 | 4.70 |
| | s024 | 15 | <0.08 | 3 055 | 6.45 |
| | s025 | 28 | <0.08 | 7 861 | >8.00 |
| | s026 | 27 | <0.08 | 3 592 | 7.14 |
| | s027 | 11 | <0.08 | 2 022 | 3.62 |
| Patients under | c001 | — | — | 541 | 0.97 |
| chemotherapy | c002 | 36 | <0.08 | 837 | 1.51 |
| | c003 | — | — | 1 614 | 3.02 |
| | c004 | 3 | <0.08 | 1 777 | 3.35 |
| | c005 | — | — | 2 257 | 4.39 |
| | c006 | 2 | <0.08 | 2 648 | 5.29 |
| | c007 | 63 | 0.11 | 3 946 | >8.00 |
| | c008 | — | — | 4 614 | >8.00 |
| | c009 | 37 | <0.08 | 5 772 | >8.00 |
| | c010 | 6 | <0.08 | 6 881 | >8.00 |
| | c011 | 46 | <0.08 | 3 122 | 6.63 |
| | c012 | 106 | 0.19 | 2 219 | 4.41 |
| | c013 | 13 | <0.08 | 2 376 | 4.77 |
| | c014 | 27 | <0.08 | 51 | 0.09 |
| | c015 | 79 | 0.14 | 108 | 0.20 |
| | c016 | 27 | <0.08 | 5 869 | >8.00 |

FIG. 1 shows these same IFNγ assay results, in graphical and statistical form.

EXAMPLE 2: Stimulation of whole blood from healthy donors and liver transplant patients.

### Origin of the Blood Samples Analyzed

The whole blood samples used in this example come from a cohort of volunteers enrolled in the EdMonHG clinical trial (ClinicalTrials. gov Identifier: NCT03995537), including:
11 healthy adult volunteers (i.e. with no symptoms of immunodeficiency); and
19 patients undergoing liver transplantation and on immunosuppressive therapy. For each of these patients, a blood sample was collected before transplantation (samples noted Pre_TH), and then every week following transplantation, for one month (samples noted successively D1-7, D8-14, D15-21 and D22-31).

### Sample Stimulation and Assay of IFNγ Secretion After Stimulation

Whole blood samples were stimulated with PHA following the same stimulation protocol as described previously.

On conclusion of 3 hours 30 minutes of stimulation, the IFNγ present in the reaction medium was assayed following the same assay protocol as described previously.

### Results

The results obtained are collated in Table 2 hereinbelow, in which INFγ production after stimulation (and without stimulation), is expressed in terms of the fluorescence intensity recorded (RFV, for “Relative Fluorescence Value”) and the estimated IFNγ concentration.

TABLE 2
Assaying IFNγ without and after PHA-P stimulation, on
whole blood from healthy donors and liver transplant patients.

| | | | Basal level | | |
|---|---|---|---|---|---|
| | | | of IFNγ | IFNγ after stimulation | |
| | Samples | | (RFV) | (RFV) | (IU/mL) |
| Healthy | s001′ | | 11 | 6703 | >8.00 |
| individuals | s002′ | | 18 | 8606 | >8.00 |
| | s003′ | | 6 | 8016 | >8.00 |
| | s004′ | | 13 | 12262 | >8.00 |
| | s005′ | | 9 | 7391 | >8.00 |
| | s006′ | | 11 | 10099 | >8.00 |
| | s007′ | | 15 | 10488 | >8.00 |
| | s008′ | | 9 | 5725 | >8.00 |
| | s009′ | | 13 | 5664 | >8.00 |
| | s010′ | | 7 | 7271 | >8.00 |
| | s011′ | | 6 | 7026 | >8.00 |
| Grafted | g001 | PreT | 58 | 1699 | 2.72 |
| patients | | D 1-7 | 18 | 1489 | 2.36 |
| | | D 8-14 | 44 | 1078 | 1.72 |
| | | D 15-21 | 31 | 3344 | 6.04 |
| | | D 22-31 | 29 | 3048 | 5.40 |
| | g002 | PreT | 28 | 10088 | >8.00 |
| | | D 1-7 | 30 | 2246 | 3.80 |
| | | D 8-14 | 67 | 2910 | 4.97 |
| | | D 15-21 | 22 | 2780 | 4.85 |
| | | D 22-31 | 48 | 2405 | 4.10 |
| | g003 | PreT | 62 | 3618 | 6.65 |
| | | D 1-7 | 103 | 1201 | 1.87 |
| | | D 8-14 | 67 | 5221 | >8.00 |
| | | D 15-21 | 15 | 3676 | 6.58 |
| | | D 22-31 | 55 | 3422 | 6.04 |
| | g004 | PreT | 20 | 4932 | >8.00 |
| | | D 1-7 | 70 | 310 | 0.47 |
| | | D 8-14 | 17 | 5495 | >8.00 |
| | | D 15-21 | 19 | 3043 | 5.39 |
| | | D 22-31 | 73 | 3168 | 5.66 |
| | g005 | PreT | 16 | 6761 | >8.00 |
| | | D 1-7 | 33 | 2726 | 4.61 |
| | | D 8-14 | 15 | 5756 | >8.00 |
| | | D 15-21 | 9 | 2631 | 4.55 |
| | | D 22-31 | 10 | 2627 | 4.54 |
| | g006 | PreT | 16 | 3874 | 7.24 |
| | | D 1-7 | 36 | 3504 | 6.21 |
| | | D 8-14 | 13 | 791 | 1.21 |
| | | D 15-21 | 15 | 3324 | 5.99 |
| | | D 22-31 | 17 | 2884 | 5.06 |
| | g007 | PreT | — | — | — |
| | | D 1-7 | 80 | 258 | 0.39 |
| | | D 8-14 | 26 | 3633 | 6.49 |
| | | D 15-21 | 53 | 2399 | 4.09 |
| | | D 22-31 | 14 | 3703 | 6.84 |
| | g008 | PreT | — | — | — |
| | | D 1-7 | 56 | 782 | 1.20 |
| | | D 8-14 | 14 | 1351 | 2.12 |
| | | D 15-21 | 17 | 2821 | 4.93 |
| | | D 22-31 | 34 | 3118 | 5.55 |
| | g009 | PreT | — | — | — |
| | | D 1-7 | 12 | 1244 | 1.95 |
| | | D 8-14 | 12 | 1808 | 2.91 |
| | | D 15-21 | 331 | 3083 | 5.48 |
| | | D 22-31 | 20 | 1469 | 2.38 |
| | g010 | PreT | — | — | — |
| | | D 1-7 | 39 | 637 | 0.97 |
| | | D 8-14 | 53 | 3698 | 6.83 |
| | | D 15-21 | | 1983 | |
| | | D 22-31 | 29 | 1842 | 3.05 |
| | g011 | PreT | — | — | — |
| | | D 1-7 | 136 | 387 | 0.60 |
| | | D 8-14 | 6 | 3228 | 5.79 |
| | | D 15-21 | 17 | 4174 | 7.96 |
| | | D 22-31 | 52 | 5086 | >8.00 |
| | g012 | PreT | — | — | — |
| | | D 1-7 | 23 | 1428 | 2.31 |
| | | D 8-14 | 18 | 3952 | 7.43 |
| | | D 15-21 | — | — | — |
| | | D 22-31 | — | — | — |
| | g013 | PreT | — | — | — |
| | | D 1-7 | 15 | 2375 | 4.04 |
| | | D 8-14 | 286 | 3969 | 7.47 |
| | | D 15-21 | — | — | — |
| | | D 22-31 | — | — | — |
| | g014 | PreT | — | — | — |
| | | D 1-7 | 8 | 1646 | 2.69 |
| | | D 8-14 | 20 | 4274 | >8.00 |
| | | D 15-21 | — | — | — |
| | | D 22-31 | — | — | — |
| | g015 | PreT | — | — | — |
| | | D 1-7 | 17 | 2213 | 3.73 |
| | | D 8-14 | — | — | — |
| | | D 15-21 | — | — | — |
| | | D 22-31 | — | — | — |
| | g016 | PreT | — | — | — |
| | | D 1-7 | 12 | 3480 | 6.34 |
| | | D 8-14 | — | — | — |
| | | D 15-21 | — | — | — |
| | | D 22-31 | — | — | — |
| | g017 | PreT | — | — | — |
| | | D 1-7 | 17 | 1202 | 1.92 |
| | | D 8-14 | — | — | — |
| | | D 15-21 | — | — | — |
| | | D 22-31 | — | — | — |
| | g018 | PreT | — | — | — |
| | | D 1-7 | 67 | 1156 | 1.85 |
| | | D 8-14 | — | — | — |
| | | D 15-21 | — | — | — |
| | | D 22-31 | — | — | — |
| | g019 | PreT | — | — | — |
| | | D 1-7 | 39 | 2994 | 5.39 |
| | | D 8-14 | — | — | — |
| | | D 15-21 | — | — | — |
| | | D 22-31 | — | — | — |

FIG. 2 shows these same IFNγ assay results, in graphical and statistical form.

### Example 3: Stimulation of Whole Blood From Healthy Donors and Patients After Septic Shock

### Origin of the Blood Samples Analyzed

The whole blood samples used in this example come from 11 healthy volunteers and 22 patients admitted to the intensive care unit of the Hôpital Edouard Herriot in Lyon (France), following septic shock.

For each of these patients monitored after septic shock, a first blood sample was produced on the day of admission or the following day, and then, if possible, a second sample on days 3-4, and finally on days 5-8 (samples noted successively as D1-2, D3-4, D5-8). Four of these patients died during this period or shortly afterwards.

### Sample Stimulation and Assay of IFNγ Secretion After Stimulation

Whole blood samples were stimulated with PHA following the same stimulation protocol as described previously.

On conclusion of 3 hours 30 minutes of stimulation, the IFNγ present in the reaction medium was assayed following the same assay protocol as described previously.

### Results

The results obtained are collated in Table 3 hereinbelow, in which INFγ production after stimulation (and without stimulation), is expressed in terms of the fluorescence intensity recorded (RFV, for “Relative Fluorescence Value”) and the estimated IFNγ concentration.

TABLE 3
Assaying IFNγ without and after PHA-P stimulation, on whole
blood from healthy donors and patients following septic shock.

| | | | Basal level | IFNγ after stimulation | |
|---|---|---|---|---|---|
| | Samples | | of IFNγ (RFV) | RFV | IU/mL |
| Healthy | s001′ | | 11 | 6703 | >8.00 |
| individuals | s002′ | | 18 | 8606 | >8.00 |
| | s003′ | | 6 | 8016 | >8.00 |
| | s004′ | | 13 | 12262 | >8.00 |
| | s005′ | | 9 | 7391 | >8.00 |
| | s006′ | | 11 | 10099 | >8.00 |
| | s007′ | | 15 | 10488 | >8.00 |
| | s008′ | | 9 | 5725 | >8.00 |
| | s009′ | | 13 | 5664 | >8.00 |
| | s010′ | | 7 | 7271 | >8.00 |
| | s011′ | | 6 | 7026 | >8.00 |
| Patients after | sep001 | D 1/D 2 | 156 | 597 | 0.91 |
| septic shock | (died) | D 3/D 4 | 27 | 1021 | 1.43 |
| | | D 5/D 8 | — | — | — |
| | sep002 | D 1/D 2 | 24 | 927 | 1.43 |
| | (died) | D 3/D 4 | 34 | 4541 | >8.00 |
| | | D 5/D 8 | — | — | — |
| | sep003 | D 1/D 2 | 7 | 1691 | 2.77 |
| | (died) | D 3/D 4 | 9 | 4499 | >8.00 |
| | | D 5/D 8 | 34 | 4383 | >8.00 |
| | sep004 | D 1/D 2 | 38 | 111 | 0.17 |
| | (died) | D 3/D 4 | 15 | 229 | 0.35 |
| | | D 5/D 8 | 29 | 481 | 0.74 |
| | sep005 | D 1/D 2 | 9 | 194 | 0.30 |
| | | D 3/D 4 | 25 | 84 | 0.13 |
| | | D 5/D 8 | 19 | 96 | 0.14 |
| | sep006 | D 1/D 2 | 29 | 692 | 1.08 |
| | | D 3/D 4 | 43 | 2617 | 4.63 |
| | | D 5/D 8 | 55 | 7040 | >8.00 |
| | sep007 | D 1/D 2 | 121 | 1396 | 2.23 |
| | | D 3/D 4 | — | — | — |
| | | D 5/D 8 | 27 | 1090 | 1.72 |
| | sep008 | D 1/D 2 | 17 | 1076 | 1.69 |
| | | D 3/D 4 | 9 | 3054 | 5.34 |
| | | D 5/D 8 | 17 | 3307 | 1.84 |
| | sep009 | D 1/D 2 | 17 | 8515 | >8.00 |
| | | D 3/D 4 | 9 | 7510 | >8.00 |
| | | D 5/D 8 | — | — | — |
| | sep010 | D 1/D 2 | 14 | 937 | 1.45 |
| | | D 3/D 4 | 7 | 1077 | 1.71 |
| | | D 5/D 8 | 9 | 2206 | 3.72 |
| | sep011 | D 1/D 2 | 7 | 3391 | 6.14 |
| | | D 3/D 4 | 7 | 7209 | >8.00 |
| | | D 5/D 8 | 35 | 7960 | >8.00 |
| | sep012 | D 1/D 2 | 78 | 5853 | >8.00 |
| | | D 3/D 4 | 100 | 4747 | >8.00 |
| | | D 5/D 8 | — | — | — |
| | sep013 | D 1/D 2 | 9 | 6031 | >8.00 |
| | | D 3/D 4 | 12 | 3726 | 6.92 |
| | | D 5/D 8 | 31 | 1991 | 3.32 |
| | sep014 | D 1/D 2 | 1 | 2555 | 4.40 |
| | | D 3/D 4 | 6 | 7475 | >8.00 |
| | | D 5/D 8 | — | — | — |
| | sep015 | D 1/D 2 | 31 | 637 | 1.00 |
| | | D 3/D 4 | 20 | 1853 | 3.07 |
| | | D 5/D 8 | — | — | — |
| | sep016 | D 1/D 2 | 55 | 3139 | 5.52 |
| | | D 3/D 4 | — | — | — |
| | | D 5/D 8 | — | — | — |
| | sep017 | D 1/D 2 | 7 | 7945 | >8.00 |
| | | D 3/D 4 | 16 | 10658 | >8.00 |
| | | D 5/D 8 | 9 | 8282 | >8.00 |
| | sep018 | D 1/D 2 | 12 | 154 | 0.23 |
| | | D 3/D 4 | 30 | 348 | 0.53 |
| | | D 5/D 8 | 6 | 625 | 0.90 |
| | sep019 | D 1/D 2 | 13 | 511 | 0.79 |
| | | D 3/D 4 | 132 | 1245 | 1.97 |
| | | D 5/D 8 | — | — | — |
| | sep020 | D 1/D 2 | — | — | — |
| | | D 3/D 4 | — | — | — |
| | | D 5/D 8 | 26 | 6541 | >8.00 |
| | sep021 | D 1/D 2 | — | — | — |
| | | D 3/D 4 | 32 | 5893 | >8.00 |
| | | D 5/D 8 | 24 | 4924 | >8.00 |
| | sep022 | D 1/D 2 | — | — | — |
| | | D 3/D 4 | 13 | 5168 | >8.00 |
| | | D 5/D 8 | 9 | 2494 | 4.16 |

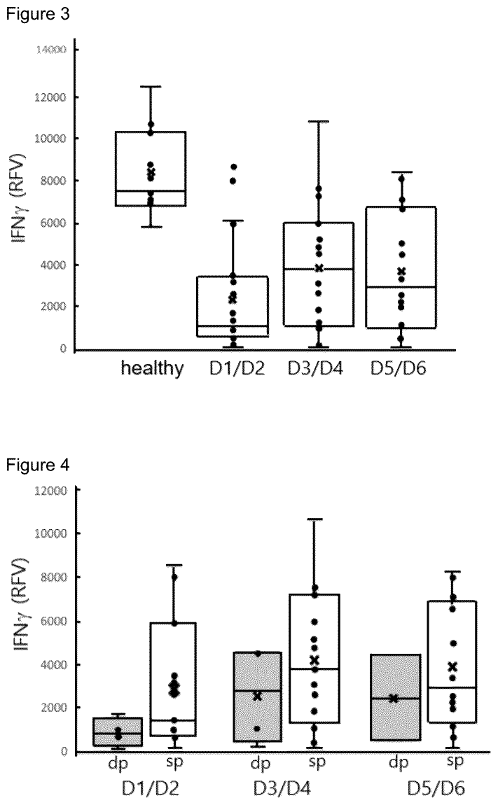

FIG. 3 shows all these IFNγ assay results, in graphical and statistical form.

FIG. 4 shows the results of IFNγ assaying after stimulation, in graphical and statistical form, differentiating between data associated with patients alive (sp) during the follow-up week, and those associated with patients who died (dp) during this follow-up period or shortly afterwards.

1. A process for determining an individual's immune status, comprising the following steps:
providing a volume of whole blood sample from said individual;
stimulating said whole blood sample by incubating it with an amount of phytohemagglutinin (PHA), at a temperature of between 35° C. and 39° C., for a minimum period of 3 hours;
evaluating the level of IFNγ production induced by this incubation/stimulation; said level thus evaluated gives an indication of the immune status of said individual.

2. The process as claimed in claim 1, in which said minimum period is 3 hours 30 minutes.

3. The process as claimed in claim 1 or 2, in which the duration of the stimulation/incubation step is between 3 hours and 8 hours.

4. The process as claimed in claim 1 or 2, in which the duration of the stimulation/incubation step is between 3 hours 30 minutes and 6 hours.

5. The process as claimed in any one of the preceding claims, in which the stimulation/incubation step is performed with phytohemagglutinin P (PHA-P).

6. The process as claimed in any one of the preceding claims, in which the stimulation/incubation step is performed with an amount of PHA at least equal to 20 μg per mL of whole blood.

7. The process as claimed in any one of the preceding claims, in which the stimulation/incubation step is performed with about 40 μg PHA per mL of whole blood.

8. The process as claimed in any one of the preceding claims, in which the temperature of the stimulation/incubation step is of the order of 37° C.

9. The process as claimed in any one of the preceding claims, in which the IFNγ production is evaluated by assaying the IFNγ by means of an immunoassay technique.

10. The process as claimed in any one of the preceding claims, in which the IFNγ production is evaluated by assaying IFNγ by means of an ELFA assay.

11. The process as claimed in any one of the preceding claims, in which the whole blood sample is heparinized.

12. Use of a process for determining an individual's immune status as claimed in any one of claims 1 to 11, for the purpose of detecting any immune deficiency.
